(19) United States
(12) Patent Application Publication
(10) Pub. No.: US 2023/0320897 A1
(43) Pub. Date: Oct. 12, 2023

(54) AUTOMATED PHACOEMULSIFICATION

(71) Applicant: JOHNSON & JOHNSON SURGICAL VISION, INC., Irvine, CA (US)

(72) Inventors: Abraham Hajishah, Irvine, CA (US); Katrina Lieu, Irvine, CA (US); Edith Fung, Diamond Bar, CA (US); Deep Mehta, Irvine, CA (US); Hajime Minoguchi, Irvine, CA (US)

(21) Appl. No.: 18/334,305

(22) Filed: Jun. 13, 2023

Related U.S. Application Data
(63) Continuation-in-part of application No. 17/634,426, filed on Feb. 10, 2022.
(60) Provisional application No. 62/888,296, filed on Aug. 16, 2019.

(51) Int. Cl. A61F 9/007; A61M 1/00

(52) U.S. Cl. CPC A61F 9/00745; A61M 1/74

(57) ABSTRACT

A computer-based automated phacoemulsification method is disclosed. The method includes determining a vacuum measurement based on a reading from a sensor coupled with an aspiration line; comparing the vacuum measurement to the at least one vacuum threshold; and providing an ultrasound power based on the comparison of the vacuum measurement to the at least one vacuum threshold.

### CROSS-REFERENCE TO RELATED PATENT APPLICATIONS

This application is a continuation-in-part of and claims priority to U.S. patent application Ser. No. 17/634,426, filed Feb. 10, 2022, which is a U.S. national stage of and claims the benefit under 35 U.S.C. § 371 of International Patent Application No. PCT/IB2020/057591, filed Aug. 12, 2020, which claims priority to U.S. Provisional Application No. 62/888,296, filed Aug. 16, 2019, all of which are incorporated herein by reference in their entirety.

### FIELD OF INVENTION

This invention generally relates to surgical systems used in ocular surgery and more specifically to an automated phacoemulsification system in ocular surgery.

### BACKGROUND

During eye surgery, such as cataract surgery, the surgeon controls various surgical system parameters. These parameters may include at least aspiration, vacuum, and ultrasound power, wherein ultrasound power controls the handpiece tip oscillation. Typically, the surgeon controls the parameters via a foot pedal. The surgeon may move the foot pedal to one of three zones, wherein a first zone may correspond to irrigation, a second zone may correspond to irrigation and aspiration, and a third zone may correspond to irrigation, aspiration, and ultrasound power. The specific parameters that are controllable depend on the zone of the foot pedal.

The level or amount of a parameter in use would correlate to the pitch of the foot pedal treadle in linear or panel mode, depending on what the user (e.g., surgeon) has specified on the phacoemulsification system. How much surgeons modulate the foot pedal, and the amount of aspiration/vacuum/power used varies and depends on surgical techniques, cataract grade, and progress or stage of the cataract procedure. Most surgeons exercise their skills, techniques, and experiences by controlling the foot pedal to navigate through the surgery. There is not a fixed procedure to follow. Each surgeon may have his/her own method to approach the surgery.

However, the surgeon may unknowingly use too much energy during the surgery. High energy may cause damage to the corneal endothelial cells and lead to corneal decompensation. Excessive heat build-up from too much power applied in the eye during surgery could also lead to corneal burns or damage other delicate ocular structures. As such, it would be desirable to have a phacoemulsification system that assists surgeons with applying ultrasound power and aspiration effectively and efficiently with enhanced precision. It would further be desirable to have a phacoemulsification system that applies power efficiently to break up lens particles by applying minimal power for smaller pieces of lens and applying maximum power for larger and occluded pieces of lens. Additionally, it would be beneficial to enhance the ergonomics of operating the phacoemulsification system by allowing surgeons to hold their foot in one position, e.g., all the way down on the foot pedal, during surgery while still receiving an appropriate amount of ultrasound power and/or aspiration for efficient cataract removal. The surgeon would not need to feather the foot pedal to achieve the desired control of the system. Moreover, a phacoemulsification system with the described invention may also minimize the learning curve for new surgeons and enhance their ability to achieve desired results.

### SUMMARY

A computer-based automated phacoemulsification method is disclosed. The method may include determining a vacuum measurement based on a reading from a sensor coupled to an aspiration line; comparing the vacuum measurement to the at least one vacuum threshold; and providing an ultrasound power based on the comparison of the vacuum measurement to the at least one vacuum threshold.

A surgical system for automated phacoemulsification is disclosed. The surgical system may include a handpiece, an aspiration line, a sensor, and a surgical console. The aspiration line may be connected to the handpiece. The sensor is communicatively coupled with the aspiration line. The surgical console may be communicatively coupled to the handpiece. The surgical console may be configured to provide a predetermined ultrasound power to the handpiece based on a comparison of a measured vacuum level and at least one vacuum threshold. The measured vacuum level may be based on a reading from the sensor.

### BRIEF DESCRIPTION OF THE DRAWINGS

The following detailed description should be read with reference to the drawings, in which like elements in different drawings are identically numbered. The drawings, which are not necessarily to scale, depict selected examples and are not intended to limit the scope of the invention. The detailed description illustrates by way of example, not by way of limitation, the principles of the invention. This description will clearly enable one skilled in the art to make and use the invention, and describes several examples, adaptations, variations, alternatives, and uses of the invention, including what is presently believed to be the best mode of carrying out the invention.

FIG. 1 illustrates an exemplary phacoemulsification system in a functional block diagram;

FIG. 2A to 2B are perspective views of an exemplary foot pedal;

FIG. 2C is a side view of an exemplary foot pedal;

FIGS. 3 to 5 are a graphical depiction of the operation of an automated phacoemulsification system;

FIG. 6 is a graphical depiction of adaptive vacuum thresholds of an automated phacoemulsification system;

FIG. 7A is a graphical depiction of a first example of a “power reset” function of the automated phacoemulsification system;

FIG. 7B is a graphical depiction of a second example of the “power reset” function of the automated phacoemulsification system;

FIG. 7C is a graphical depiction of a third example of the “power reset” function of the automated phacoemulsification system;

FIG. 8 illustrates an exemplary graphical user interface (GUI);

FIG. 9 illustrates an exemplary graphical user interface (GUI);

FIG. 10 is a flow chart of a computer-based automated phacoemulsification method; and

FIG. 11 illustrates an exemplary anti-vacuum surge device (AVS).

### DETAILED DESCRIPTION OF THE PREFERRED EXAMPLES

Automated phacoemulsification systems are disclosed. The automated phacoemulsification systems disclosed herein are designed to assist surgeons with applying ultrasound power and aspiration to a handpiece effectively and efficiently such that minimal energy is used during phacoemulsification. The systems are intended to replicate or perform similarly to the patterns and motions of a surgeon using a foot pedal to control these parameters with enhanced precision. The systems integrate the expertise of skilled surgeons with advanced computer technology. The systems eliminate the need of the foot pedal to control ultrasound power and aspiration during the phacoemulsification portion of cataract surgery. Instead, these parameters are controlled and applied via computer technology.

The systems described herein apply power efficiently to break up lens particles by applying minimal power for smaller pieces and applying maximum power for larger and occluded pieces, while incorporating key concepts of followability and holdability. Followability may be defined as the ability to attract lens particles to the phacoemulsification tip located on the handpiece. Followability may rely on the overall fluidics behavior of the system, and particularly, the maximum aspiration setpoint of the system. Holdability may be defined as how well a lens particle is held or stays at the phacoemulsification tip. Holdability may rely on the overall fluidics behavior of the system and especially the maximum vacuum setpoint of the system.

The systems provide efficiency by minimizing foot pedal “feathering” action between different foot pedal zones. A foot pedal of phacoemulsification systems may include three foot pedal positions—position 1 (FP1), position 2 (FP2) and position 3 (FP3) that correspond to the three zones described below.

FP1, which corresponds to the first zone, may provide irrigation. FP2, which corresponds to the second zone, may provide aspiration. FP3, which corresponds to the third zone, may provide ultrasound/phacoemulsification. Each foot pedal position may be additive, so FP2 provides irrigation and aspiration and FP3 provides irrigation, aspiration, and ultrasound/phacoemulsification.

With traditional phacoemulsification systems (those systems that do not include automated phacoemulsification), a user, such as a surgeon or operator, may generally be in FP2 to apply irrigation and aspiration. When a lens particle is at the tip, the user may go into FP3 to apply enough power to break up the lens particle into small pieces and extract them from the eye. When the lens particle has been sufficiently broken up, the user may return to FP2 to pull another lens particle to the phacoemulsification tip, press down into FP3 again to apply power to break it up, and repeat. The user may continue feathering between FP2 and FP3 until all lens particles have been removed.

In the automated phacoemulsification system described herein, feathering may not be needed because the surgeon may only need to stay in FP3 (the pitch in FP3 is irrelevant) for the algorithm to start and apply power and aspiration based on the vacuum level relative to the algorithm-defined vacuum thresholds. As the surgeon works to bring a particle to the tip, the automated phacoemulsification system will compare vacuum levels and determine when to apply power. In one example, the automated phacoemulsification system may apply a lower level of power when there is a particle around the tip but not necessarily fully occluding it, and higher level of power when the particle is fully blocking and/or occluding the tip. Therefore, the automated phacoemulsification may not need to move between FP2 and FP3 to titrate how much power or vacuum the system needs. This increased efficiency may reduce ultrasound time and the total amount of energy used during surgery which may decrease patient risk during the procedure.

By using as little energy as possible, the systems disclosed herein help eliminate or reduce chances of damage to the corneal endothelial cells as excessive damage can lead to corneal decompensation. Excessive heat build-up from too much power being applied in the eye can also lead to corneal burns and/or damage other delicate ocular structures. The automated systems disclosed reduce heat buildup by providing the appropriate and most efficient amount of ultrasound power.

In addition, the algorithm may allow for additional functionality to be added to FP3 if a surgeon desires additional control of one or more parameters or functions of the system. For example, a surgeon is in FP3 with the automated phacoemulsification algorithm activated and she decides she wants more power quickly, then the user may make a double foot tapping motion within FP3 to generate more power or activate some sort of power boost (e.g., 105% x Max Power Setpoint). This could allow the surgeon to get more power quickly without having to wait for the scrub nurse to change the power parameters on the GUI. In addition, if the foot pedal is a single linear pedal where a yaw capability is not available an additional functionality of the foot pedal is provided.

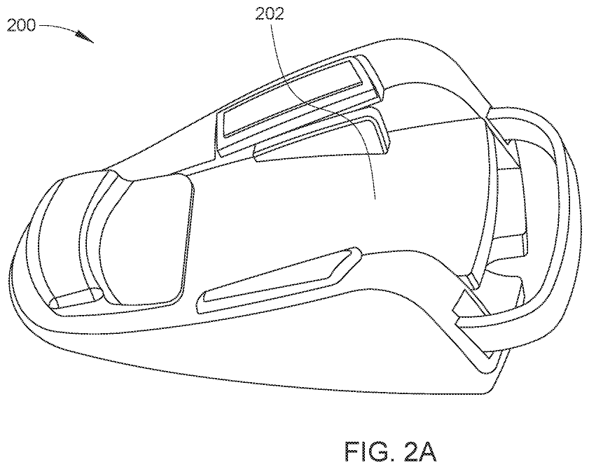

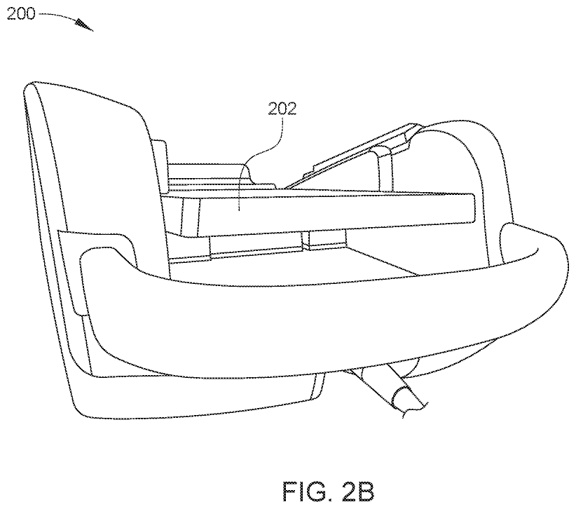

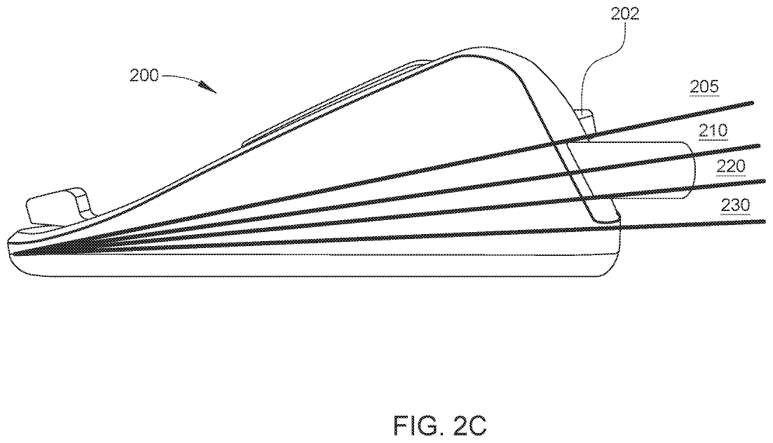

In one example of a double foot tapping motion, the user is in automated phacoemulsification mode in FP3 with the foot pedal (see FIG. 2A to 2C) pushed all the way down (i.e., 100% of PF3) and desires a temporary power boost (e.g., 105% power of the max power setpoint). In this instance, the user may lift their foot to about 80% or less within FP3 and then quickly press the treadle all the way back down. The user may repeat this motion again quickly (i.e., double tap motion). This double tap may result in a temporary power boost, which may last between 1 to 5 seconds. After the power boost, the power level may return back to its original set point.

In another example of a double foot tapping motion, the user may be hovering in automated phacoemulsification mode in FP3 (e.g., 50% of FP3). In this scenario, if the user desires a power boost, they may quickly press the treadle all the way down in FP3 two times (i.e., double tap motion). This double tap may result in a temporary power boost, which may last between 1 to 5 seconds. After the power boost, the power level may return back to its original set point. The double tap may activate the power boost if the foot pedal quickly passes from 80% of FP3 to 100% of FP3 two times (e.g., within msec).

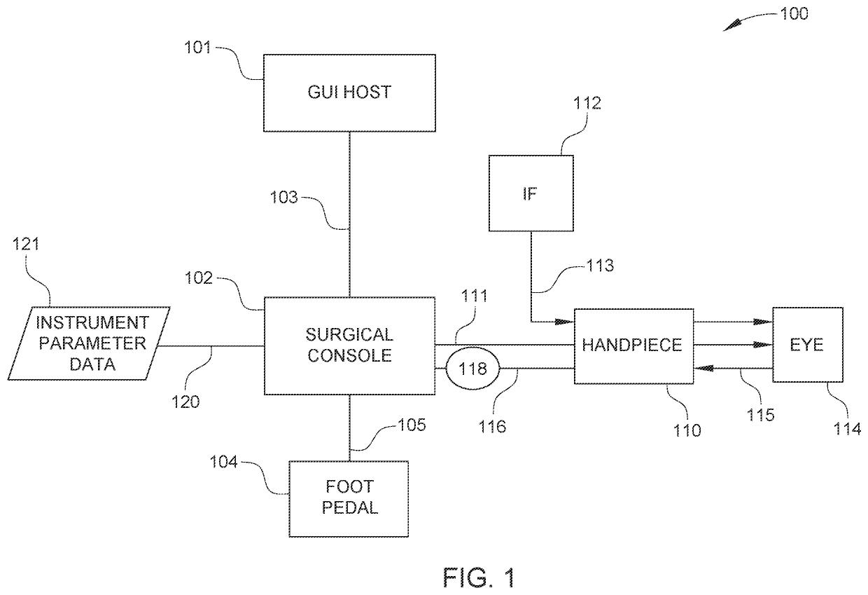

FIG. 1 illustrates an exemplary phacoemulsification system 100 in a functional block diagram that may be employed in accordance with an aspect of the present disclosure. A serial communication cable 103 connects GUI host 101 module and surgical console 102 module for the purposes of controlling the surgical console 102 by the GUI host 101. GUI host 101 and instrument host 102, as well as any other component of system 100, may be connected wirelessly. Surgical console 102 may be considered a computational device in the arrangement shown, but other arrangements are possible. An interface communications cable 120 is connected to surgical console 102 module for distributing instrument parameter/sensor data 121, and may include distribution of instrument settings and parameters information, to other systems, subsystems and modules within and external to surgical console 102 module. Although shown connected to the surgical console 102 module, interface communications cable 120 may be connected or realized on any other subsystem (not shown) that could accommodate such an interface device able to distribute the respective data.

A switch module associated with a foot pedal 104 may transmit control signals relating internal physical and virtual switch position information as input to the surgical console 102 over serial communications cable 105. The foot pedal 104 may be connected wirelessly (e.g., Bluetooth, infrared, etc. to the surgical console 102. Surgical console 102 may provide a database file system for storing configuration parameter values, programs, and other data saved in a storage device (not shown). In addition, the database file system may be realized on the GUI host 101 or any other subsystem (not shown) that could accommodate such a file system.

The phacoemulsification system 100 has a handpiece 110 that includes a needle and electrical means, typically a piezoelectric crystal, for ultrasonically vibrating the needle. The surgical console 102 supplies ultrasound power 111 to the handpiece 110. An irrigation fluid source 112 can be fluidly coupled with/to handpiece 110 through line 113 via a sleeve (not shown) that at least partially surrounds the needle and includes at least one port for delivery of the irrigation fluid by the handpiece 110 to an eye, or affected area or region, indicated diagrammatically by block 114. Alternatively, the irrigation source may be routed to eye 114 through a separate pathway independent of the handpiece 110 using a bimanual technique known in the art. The surgical console 102 controls one or more pumps. The one or more pumps provide aspiration applied to the handpiece and eye through lines 115 and 116, respectively. A surgeon/operator may select system parameters using the handpiece, foot pedal, via the instrument host and/or GUI host, and/or by voice command.

The phacoemulsification system 100 may include a sensor system. For example, the system 100 may include at least one sensor 118 coupled anywhere along the aspiration line 116. In an example, one or more sensors may be located in the handpiece 110, the console 102, and/or coupled anywhere along the aspiration line 116. Although not shown, the system 100 may also include at least one sensor coupled anywhere along the irrigation line 113. Measurements and/or data from the at least one sensor 118 may be communicated to the surgical console 102.

The surgical console 102 generally comprises at least one processor board. Surgical console 102 may include many of the components of a personal computer, such as a data bus, a memory, input and/or output devices (including a touch screen (not shown)), and the like. Surgical console 102 will often include both hardware and software, with the software typically comprising machine readable code or programming instructions for implementing one, some, or all of the methods described herein. The code may be embodied by a tangible media such as a memory, a magnetic recording media, an optical recording media, or the like.

A controller (not shown) may have (or be coupled with/to) a recording media reader, or the code may be transmitted to surgical console 102 by a network connection such as an internet, an intranet, an Ethernet, a wireless network, or the like. Along with programming code, instrument host 102 may include stored data for implementing the methods described herein, and may generate and/or store data that records parameters reflecting the treatment of one or more patients.

FIGS. 2A to 2B show perspective views of a foot pedal 200 that may be employed in accordance with an aspect of the present disclosure. The foot pedal 200 may be moved by a user's foot to designate one of three different zones 205 (FP0 as described below), 210 (FP1 as described above), 220 (FP2 as described above), and 230 (FP3 as described above). Zone 205 (FP0) may designate a default starting position.

In an example, the user may press the foot pedal towards the floor to change zones. For example, if the user is in the first zone 210, they can press the foot pedal towards the floor to change to the second zone 220 (similar to how a driver presses the accelerator to increase a vehicles speed). In some examples, the thresholds (e.g., amount of force required) to change from one zone to another zone may be adjusted. For example, the threshold may be adjusted via a graphical user interface on the surgical console. In addition, the available travel length of the treadle in each zone of the foot pedal may be increased or decreased and a detent or other indicator (e.g., tactile, or audible) may be placed between zones to indicate movement from one zone to another.

In one example, the first zone 210 may designate irrigation, the second zone 220 may designate irrigation and aspiration, and the third zone 230 may designate irrigation, aspiration, and ultrasound power. The corresponding parameters to each zone 210, 220, 230 may be controlled by the surgeon via the pitch of the foot pedal 200 during surgery, an operation or procedure. For example, the surgeon may control: (1) the level or amount of the irrigation parameter via the pitch of the foot pedal 200 treadle 202 in the first zone 210; (2) the level or amount of the aspiration parameter via the pitch of the foot pedal treadle 202 in the second zone 220; and (3) the level or amount of the ultrasound power parameters, such as the amount or level of power, via the pitch of the foot pedal treadle 202 in the third zone 230.

The automated phacoemulsification systems described herein may be selected and/or activated in the third zone 230. When the system is activated and the foot pedal 200 is in the third zone 230, the parameters, including ultrasound power, aspiration, and irrigation, are provided automatically by the surgical console 102 without the need of a surgeon adjusting the pitch of the foot pedal treadle 202. The automated phacoemulsification system may activate automatically when the foot pedal is in the third zone 230.

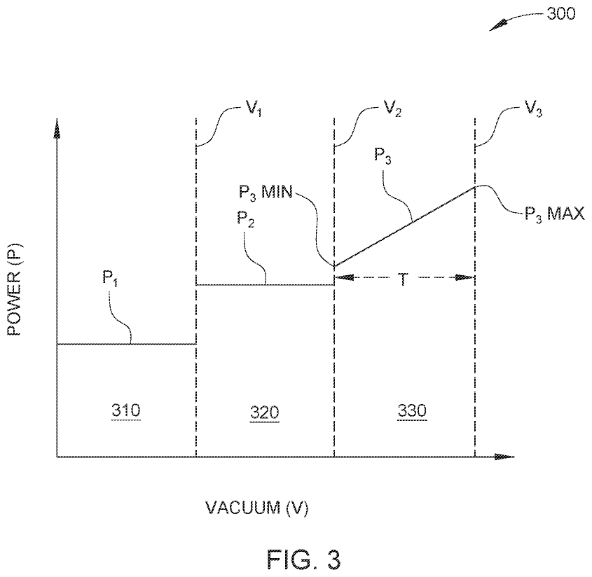

FIG. 3 is a graph 300 which depicts the operation of an automated phacoemulsification algorithm (automated phacoemulsification) for a phacoemulsification system. The system may be the system 100 depicted in FIG. 1 or any other phacoemulsification system known in the art. The operation of the system may be performed at least in part via the surgical console's 102 computer-based software. A user, such as a surgeon or operator, may select or define specific settings prior to beginning of a procedure (e.g., operation or surgery). The settings that may be defined for regular phacoemulsification mode on a phacoemulsification system may include U/S power (%) and different power configurations/modalities (e.g., Pulse, WhiteStar, etc.), aspiration (cc/min), vacuum (mmHg). For each setting, a minimum and maximum setpoint may be specified. Each setpoint may depend on the selected mode (i.e., linear or panel). For example, in linear mode, a user may need to define both a minimum and maximum setpoint, but in panel mode, only a maximum setpoint may be needed. For automated phacoemulsification, a user may select the described setpoints as needed for regular phacoemulsification mode in addition to having to select at least one additional user inputted vacuum setpoint/thresholds.

Regardless of automated phacoemulsification or regular phacoemulsification mode, the user may still define the maximum vacuum setpoint (i.e., V₃ in FIG. 3). Automated phacoemulsification may allow for at least one additional user inputted vacuum setpoints/thresholds. In one example, a user may select at least one vacuum threshold V. In another example, the vacuum thresholds V₁ and V₂ may be determined by the system where those thresholds would be lesser than the user defined maximum vacuum setpoint V₃. These vacuum thresholds V₁ and V₂ and maximum vacuum setpoint V₃ essentially define three different vacuum regions that may allow for three different U/S power levels (0-100%) to be applied depending on where the actual vacuum detected in the aspiration line falls within when the user is in FP3.

As shown in FIG. 3, a user may select a first vacuum threshold V₁, a second vacuum threshold V₂, and a maximum vacuum setpoint V₃. The second vacuum threshold V₂ may be greater than the first vacuum threshold V₁. The maximum vacuum setpoint V₃ may be greater than the second vacuum threshold V₂. Although FIG. 3 shows a system with two vacuum thresholds V₁, V₂, more or less vacuum thresholds may be selected by a user, and maximum vacuum setpoint V₃ that is user defined. Additionally, the user may select a separate vacuum threshold that triggers when to enable or activate automated phacoemulsification when the foot pedal 200 is in the third zone 230 wherein any vacuum measurement below this vacuum threshold would not activate the automated phacoemulsification even if it is enabled in the GUI settings. If automated phacoemulsification is enabled in the GUI settings, and at higher vacuum in the aspiration line during occlusions (e.g., higher than 300 mmHg), the phacoemulsification system may allow automated phacoemulsification to be active for as long as the user is in FP3, thus allowing the user to benefit from the power saving aspects of automated phacoemulsification since higher vacuum correlates to full occlusions, during which surgeons would typically use higher amounts of U/S power. An automated phacoemulsification algorithm that is a function of this separate vacuum threshold may allow users to manually apply as little power as they desire depending on the FP3 pitch since regular phacoemulsification mode is active; automated phacoemulsification algorithm would kick in only after the separate vacuum threshold is reached.

Alternatively, the user may select one vacuum threshold and the system may automatically define one or more of the other vacuum thresholds. For example, after the user has already selected the maximum vacuum setpoint V₃ (which is defined by the user regardless of automated phacoemulsification or regular phacoemulsification mode), the system would define the first and second vacuum thresholds V₁ and V₂ based on the maximum vacuum setpoint V₃. For example, the system may automatically define the second vacuum threshold V₂ as 50% of the user selected maximum vacuum setpoint V₃ and the first vacuum threshold as 50% of the second vacuum threshold V₂.

Alternatively, the system may automatically define the at least one vacuum threshold V₁, V₂, V₃ based on the vacuum levels during regular mode (i.e., when automated phacoemulsification is not activated). For example, if the maximum vacuum level during regular mode is 400 mmHg, the system may automatically define 400 mmHg as the maximum vacuum setpoint V₃. The system may further automatically define additional vacuum levels V₁, V₂ based on a percentage of the maximum vacuum setpoint V₃.

In an example, a user may toggle between regular mode and automated phacoemulsification mode via the surgical console's GUI. If enabled, the automated phacoemulsification would be active until the user decides to disable it with the toggle. After disabling the automated phacoemulsification, the system will return to regular mode operation. In another example, a user may be able to toggle between regular mode and automated phacoemulsification mode via the foot pedal. For example, the foot pedal may include a switch that the surgeon can press to toggle between regular mode and automated phacoemulsification mode.

During the operation or procedure, a vacuum measurement based on the reading from the at least one sensor 118 coupled with the aspiration line 116 is compared to the vacuum thresholds V₁, V₂, V₃. The vacuum thresholds V₁, V₂, V₃ may define sections 310, 320, 330, wherein a vacuum measurement less than the first vacuum threshold V₁ would fall into a first section 310, a vacuum measurement greater than the first vacuum threshold V₁ and less than the second vacuum threshold V₂ would fall into a second section 320, and a vacuum measurement greater than the second vacuum threshold V₂ and less than the third or maximum vacuum threshold V₃ would fall into a third section 330. These sections 310, 320, 330 may generally define different stages of occlusion. An occlusion may be defined as varying degrees of blockage of the handpiece tip by lens particles. A vacuum measurement that falls in the first section 310 may signify no occlusion. A vacuum measurement that falls in the second section 320 may signify a weak (or partial) occlusion. A vacuum measurement that falls in the third section 330 may signify a strong (or complete or almost complete) occlusion.

Prior to the operation or procedure, a user may also select the ultrasound power P₁, P₂, P₃ applied to the handpiece 110 based on the vacuum measurement and its relationship to the selected thresholds. For example, the user may define a first power P₁ for the first section 310, a second power P₂ for the second section 320, and a third power P₃ for the third section 330. Alternatively, the system algorithm may automatically define ultrasound power levels for each stage of occlusion based on the power used during regular mode phacoemulsification. For example, the defined automated phacoemulsification ultrasound powers may be based on a percentage of the regular mode ultrasound power level. Further, the power applied (i.e., P₁) prior to the first vacuum threshold (i.e., V₁) may be zero.

During the procedure, the at least one sensor 118 sends data to the surgical console 102. The sensor data is used to determine a real-time vacuum measurement. The vacuum measurement is compared to the predetermined vacuum thresholds V₁, V₂, or maximum vacuum setpoint V₃. If the vacuum measurement falls within the first section 310 (e.g., the vacuum measurement is equal to or below threshold V₁), the first power P₁ is automatically applied to the handpiece 110 via the surgical console 102. If the vacuum measurement falls within the second section 320 (e.g., the vacuum measurement passes threshold V₁, but is equal to or lower than threshold V₂), the second power P₂ is applied. If the vacuum measurement falls within the third section 330 (e.g., the vacuum measurement passes threshold V₂, but is equal to or lower than maximum vacuum setpoint V₃), the third power P₃ is applied. The powers P₁, P₂, P₃ are applied to the handpiece 110 automatically via the surgical console 102 without the user controlling the power via the pitch of the foot pedal 200 treadle.

A higher vacuum measurement represents a stronger occlusion. A stronger occlusion requires higher power to break up the particles and eliminate the occlusion. Therefore, as shown in FIG. 3, the second power P₂ may be greater than the first power P₁ and the third power P₃ may be greater than the second power P₂.

Further, the user may select more than one ultrasound power level for each section. For example, as shown in FIG. 3, the user may select a minimum third power P_{3M}TN and a maximum third power P_{3MAX}. The minimum third power P_{3MIN} and the maximum third power P_{3MAX} may be defined as percentages of the third power P₃. For example, the minimum third power P_{3MIN} may be defined as 35% of the selected third power P₃, while the maximum third power P_{3MAX} may be defined as 100% of the third power P₃.

When the vacuum measurement exceeds the second vacuum threshold V₂, the power applied P₃ to the handpiece 110 may ramp up from the minimum third power P_{3MIN} to the maximum third power P_{3MAX}. The user may select different power ramps to accommodate different lens densities. For example, a surgeon may want to ramp up power faster if they encounter a dense lens to break up the lens more quickly. In another example, a user may want ramp up power slower if they encounter a dens lens so they can ease into breaking up the lens. In another example, a user dealing with a soft lens may want to ramp up power faster to extract the particles more quickly. In another example, a user dealing with a soft lens may want to ramp up power slower and be more cautious.

The third power P₃ may either be set to panel mode or linearly increase from the minimum third power P_{3MIN} to the maximum third power P_{3MAX} over a user selected period of time T. Alternatively, the period of time T may be inherent in the system algorithm. The time T affects the slope of the function and produces different functions for different ramp times. The function may be represented as: F(X)=[(P_{3MAX}−P_{3MIN})/(T*5)]X+P_{3MIN}.

In the equation above, “5” converts time T from 200 milliseconds to seconds. Further, F(X) is the range of the minimum and maximum power (P_{3MIN}, P_{3MAX}) and X is the domain. For example, if the maximum third power P_{3MAX} is 100% and the third minimum power P_{3MIN} is 35%, the equation becomes F(X)=[(100%−35%)/(T*5)]X+35%. The range is (35%, 100%) and the domain is (0, 65).

Tables 1, 2, and 3 below show examples for ramp times T of 0.5, 1, and 2 seconds respectively when the maximum third power P_{3MAX} is 100% and the third minimum power P_{3MIN} is 35%.

If the selected ramp time is 0.5 second, the formula becomes F(X)=(13%/0.5)*X+35% or simply F(X)=26%*X+35%. Table 1 below shows the time progress and power increment at each interval when the ramp time is 0.5 seconds.

TABLE 1
Time Progress and Power Increments with a Ramp
Time of 0.5 Seconds

| Time(sec) | X | F(X) = (26%)*X + 35% | Power = F(X)*50% |
|---|---|---|---|
| (T) | (X = 5*T) | (Same as FP3%) | (Final Power) |
| 0.0 | 0 | 35% | 18% |
| 0.2 | 1 | 61% | 31% |
| 0.4 | 2 | 87% | 44% |
| 0.5 | 3 | 100% | 50% |

If the selected ramp time is 1 second, the formula becomes F(X)=(13%/1)*X+35% or simply F(X)=13%*X+35%. Table 2 below shows the time progress and power increment at each interval when the ramp time is 1 second.

TABLE 2
Time Progress and Power Increments with a Ramp
Time of a 1 Second

| Time(sec) | X | F(X) = (13%)*X + 35% | Power = F(X)*50% |
|---|---|---|---|
| (T) | (X = 5*T) | (Same as FP3%) | (Final Power) |
| 0.0 | 0 | 35% | 18% |
| 0.2 | 1 | 48% | 24% |
| 0.4 | 2 | 61% | 31% |
| 0.6 | 3 | 74% | 37% |
| 0.8 | 4 | 87% | 44% |
| 1.0 | 5 | 100% | 50% |

If the selected ramp time is 2 seconds, the formula becomes F(X)=(13%/2)*X+35% or simply F(X)=6.5%*X+35%. Table 3 below shows the time progress and power increment at each interval when the ramp time is 2 seconds.

TABLE 3
Time Progress and Power Increments with a Ramp
Time of 2 Seconds

| Time(sec) | X | F(X) = (6.5%)*X + 35% | Power = F(X)*50% |
|---|---|---|---|
| (T) | (X = 5*T) | (Same as FP3%)) | (Final Power) |
| 0.0 | 0 | 35% | 18% |
| 0.2 | 1 | 41.5% | 21% |
| 0.4 | 2 | 48% | 24% |
| 0.6 | 3 | 54.5% | 27% |
| 0.8 | 4 | 61% | 31% |
| 1.0 | 5 | 67.5% | 34% |
| 1.2 | 6 | 74% | 37% |
| 1.4 | 7 | 80.5 | 40% |
| 1.6 | 8 | 87% | 44% |
| 1.8 | 9 | 93.5% | 47% |
| 2.0 | 10 | 100% | 50% |

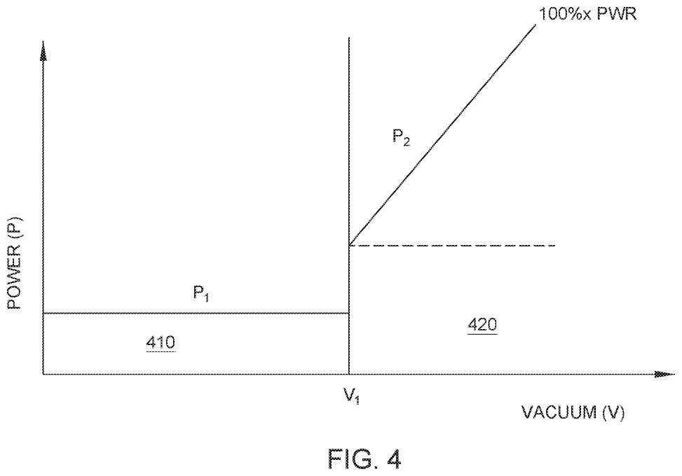

In one example, automated phacoemulsification mode may only utilize one vacuum threshold. FIG. 4 is a graph which depicts an operation of an automated phacoemulsification system with only one vacuum threshold. The system may be the system 100 depicted in FIG. 1. In one example, the user may be required to press the treadle into third zone 230 to utilize automated phacoemulsification.

As shown in FIG. 4, automated phacoemulsification mode may start in a first section 410. When operating in the first section 410, the ultrasound power P₁ may be a percentage of the max power setpoint. For example, P₁ may be 30% of the max power setpoint. P₁ may remain constant throughout the first section 410. In another example, P1 may be 0% of the max power setpoint. In an example, once the user has pressed the treadle into third zone 230, the automated phacoemulsification algorithm controls the power delivered to the needle of the handpiece based on the one or more selected parameters (e.g., threshold V₁, maximum vacuum set point, maximum power setpoint, ramp time, etc.)

When a measured vacuum level reaches or surpasses vacuum threshold V₁, the system moves from operating under the parameters or settings of the first section 410 to operating under the parameters or settings of the second section 420. The ultrasound power P₂ in the second section 420 may a be a higher percentage of the max power setpoint than in the first section 410. For example, P₁ may be 30% of the max power setpoint while P₂ may be 70% of the max power setpoint. In the second section 420, as time increases, P₂ may also increase until it reaches the max power setpoint. For example, at V₁, P₂ may be 70% of the max power setpoint, but increase to the max power setpoint over a predetermined period of time. In another example, P₁ may be 0 and P₂ may start at a certain percentage of the max power setpoint and P₂ may increase to the max power over a predetermined period of time. In another example, P₂ may increase as the vacuum level increases. In another example, P₂ may start at P₁ and increase to the max power over a predetermined period of time.

In one example, the user may not be able to set the vacuum threshold V₁. Instead, V₁ may depend on the tip size of the needle (e.g., gauge), the irrigation sleeve size, the IOP, aspiration flow, and/or the irrigation flow.

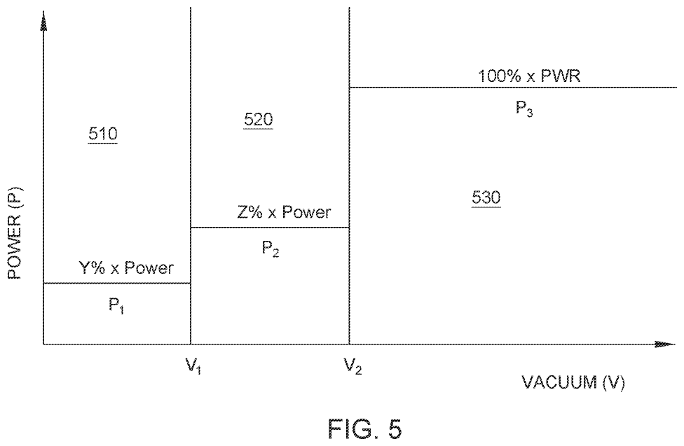

In another example, automated phacoemulsification mode may utilize two vacuum thresholds (e.g., V₁ and V₂). FIG. 5 is a graph which depicts an operation of an automated phacoemulsification algorithm with two vacuum thresholds. The system may be the system 100 depicted in FIG. 1. In one example, the user may be required to move the treadle of the foot pedal into third zone 230 to utilize automated phacoemulsification.

As shown in FIG. 5, automated phacoemulsification mode may start in a first section 510 (upon the treadle entering third zone 230). When operating in the first section 510, the ultrasound power P₁ may be a percentage of the max power setpoint. For example, P₁ may be 30% of the max power setpoint. Power P₁ may remain constant throughout the first section 510 as shown in the graph or in another example, the ultrasound power P₁ may increase linearly in the first section 510. In another example P₁ may be 0% of the max power setpoint and P₂ may start at a certain percentage of the max power setpoint. In another example, P₂ may start at zero and increase linearly to the next vacuum threshold.

When a vacuum threshold V₁ is reached or passed, the system moves from first section 510 to a second section 520. The ultrasound power P₂ in the second section 520 may a be a higher percentage of the max power setpoint than in the first section 510. In one example, P₂ may remain constant throughout the second section 520. In another example, P₂ may increase over a predetermined period of time. In yet another example, P₂ may increase as the vacuum level increases.

When a vacuum threshold V₂ is reached or passed, the system moves from the second section 520 to a third section 530. The ultrasound power P₃ in the third section 530 may a be a higher percentage of the max power setpoint than in the first section 510 and section 520. In one example, P₃ may be the max power setpoint. P₃ may remain constant throughout the third section 530. In another example, P₃ may increase over a predetermined period of time. In yet another example, P₃ may increase as the vacuum level increases.

Further, as shown in FIG. 5, power applied in each of the sections 510, 520, and 530 may be in panel mode (i.e., no power ramping).

In one example, the user may not be able to set the vacuum threshold V₁ or V₂. Instead, V₁ and/or V₂ may depend on the needle tip size and/or shape, the sleeve size and/or shape, the IOP, aspiration flow, and/or the irrigation flow.

The threshold levels discussed above may be based on real-time estimated intraocular pressure (IOP), tip size, and aspiration flow rate. Real-time IOP may be calculated by the irrigation sensor, irrigation speed, and sleeve resistance.

For example, once the tip size is determined by the system, the threshold level(s) may be determined by real-time estimated IOP, an experimentally derived second-degree polynomial equation, and experimentally derived offset using the following equation: P_{Threshold}=P_{IOP}+P_{Difference}−P_{Constant}, where P_{Difference}=−A*(F_{Aspiration})²−B*(F_{Aspiration}). P_{Threshold} may be defined as the vacuum threshold pressure. P_{IOP} may be defined as the estimated IOP. P_{Difference} may be defined at the experimentally determined pressure difference. P_{Constant} may be defined as the experimentally determined pressure offset. F_{Aspiration} may be defined as the aspiration flow rate. A may be defined as a positive constant A based on tip size. B may be defined as the positive constant B based on tip size.

Actual pressure difference and flow rate may follow a second-degree polynomial relationship due to fluid turbulence at low flow rates (Navier-Stokes equation). The two constants used to define the second-degree polynomial equation in the vacuum threshold calculation algorithm (A and B) may be determined by fitting a polynomial trendline to experimental data gathered by measuring the pressure difference between the aspiration line and the tip at various flow rates at free vacuum and partially occluded situations for threshold 1 and threshold 2, respectively. In addition to this pressure difference, a constant offset was included in the equation to account for pulsation in steady state IOP. This constant offset may be determined experimentally by averaging the peak-to-peak steady flow IOP pulsation in a rigid chamber. To calculate the vacuum threshold, pressure offset may be subtracted from, and pressure difference equation is added to the real-time estimated IOP.

Furthermore, a user may be able to select a sensitivity level for the above described automated phacoemulsification threshold levels. For example, a user may be able to select a low, medium, or high sensitivity option. Based on reaching or passing a threshold, the sensitivity level may determine how soon the power will activate when the user is operating in automated phacoemulsification. For example, if the user selects a “high” sensitivity level, the power may activate sooner at a lower vacuum level (i.e., higher aspiration pressure). If the user selects a “low” sensitivity level, the power may activate at a higher vacuum level (i.e., lower aspiration pressure).

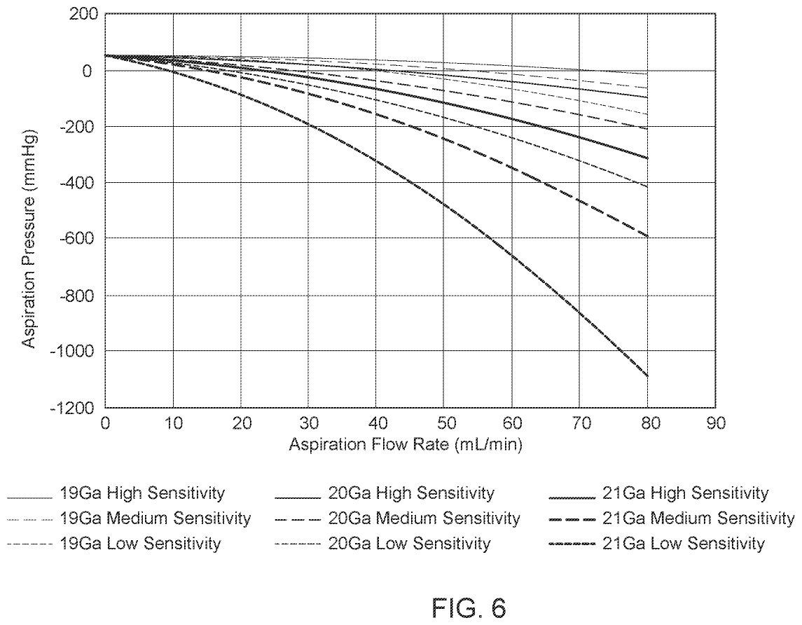

FIG. 6 is a graph that illustrates the aspiration flow for a 19 Ga tip, 20 Ga tip, and 21 Ga tip at high, medium, and low sensitivity levels. Specifically, FIG. 6 illustrates the varying or adaptive thresholds levels across different aspiration set points (ml/min) when the IOP is set to 70 mmHg for a 19 Ga tip, 20 Ga tip, and 21 Ga tip.

Further, as shown in FIG. 6, each tip size is also profiled for high, medium, and low sensitivity levels. A low sensitivity level may contain a set of higher vacuum levels/thresholds. With a low sensitivity level, the system will activate power when it detects a full occlusion. A high sensitivity level may contain a set of lower vacuum levels/thresholds. With a high sensitivity level, the system will activate power if it detects a partial occlusion.

Further, the user may select segments of the foot pedal 200 treadle to designate linear verses panel mode. For example, the user may designate the 0-50% segment of the foot pedal 200 treadle in the third zone 230 to define linear mode, and the 51-100% segment of the foot pedal 200 treadle in the third zone 230 to define panel mode.

The user may also select the power modality or configuration prior to surgery. For example, the user may select power to be either continuous, pulse (short or long pulses), or supersonics mode. The user may also use WhiteStar mode or Variable WhiteStar mode in conjunction with either continuous, pulse, or supersonics mode. In continuous mode, the power is constantly on and applied when the user is in the third zone 230. In pulse mode, the surgeon may specify the number of pulses per second. For example, the surgeon may be able to specify anywhere between 1 to 100 pulses per second. The amount of power applied in each pulse may depend on the max power setpoint. For example, in short pulse mode, the applied power may range from 1-14 pulses per second with a pulse width of 50 milliseconds (ms) (+/−5 ms). In long pulse mode, the applied power may range from 1-6 pulses per second with a pulse width of 50 ms (+/−150 ms). In supersonics mode, the applied power allows the phacoemulsification tip a motion greater than 100 KHz. In WhiteStar mode, modulated pulses of energy are delivered with brief cooling periods applied based on duty cycles expressed as pulse time on/off that is user defined. In Variable WhiteStar, different duty cycles per user selection apply modulated pulses of energy are delivered depending on the FP3 pitch. The FP3 zone is divided into four quadrants, and different duty cycles are applied depending on the quadrant.

In addition to power, a user may select aspiration levels for each section 310, 320, 330 prior to surgery. The user may select a maximum aspiration level unique to each section 310, 320, 330 to correlate to occlusion and vacuum levels. Alternatively, the system algorithm may automatically define the aspiration levels based on the aspiration applied during regular mode phacoemulsification.

The system may apply high aspiration levels or an “aspiration boost” for low or weak occlusion levels, including the first and second section 310, 320, and low aspiration levels for strong occlusion levels, including the third section 330 (FIG. 3). The aspiration boost may be used to draw particles to the tip of the handpiece 110 when then there is no or weak occlusion. The aspiration is provided automatically by the surgical console 102 based on the vacuum measurement and corresponding section 310, 320, 330. For example, when operating in section 310, the aspiration boost may provide 120% of the aspiration max setpoint when the surgeon is operating in the second zone 220 or third zone 230. In section 320, the aspiration boost may provide the 100% of the aspiration max setpoint when the surgeon is operating in third zone 230. In section 330, the aspiration boost may provide 70% of the aspiration max setpoint when the surgeon is operating in the third zone 230.

In one example, the third zone 230, as described in FIG. 2, may be split into multiple segments such that the surgeon can specify different power ramping/profiling behavior based on how far the treadle has traveled within the third zone 230 (e.g., FP3). For example, the 0-50% segment of FP3 may be defined as linear mode, while the 51-100% segment may be defined as panel mode. With respect to all examples provided herein, other power profiling/ramping behavior may also be available for user selection and/or as manufacturer settings where the power would increment accordingly per a defined maximum power setpoint (e.g., set by the user and/or manufacturer) could include linear, panel, exponential, and logarithmic.

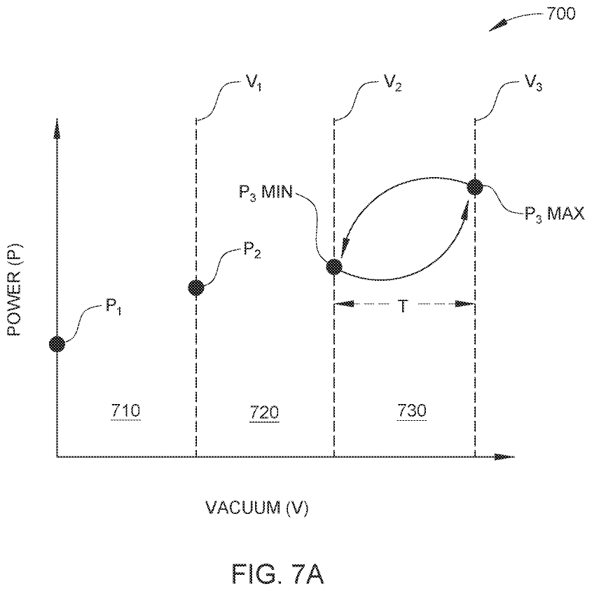

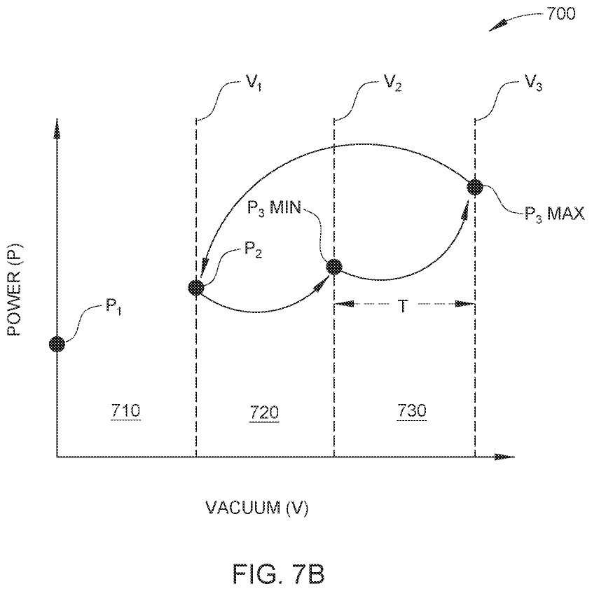

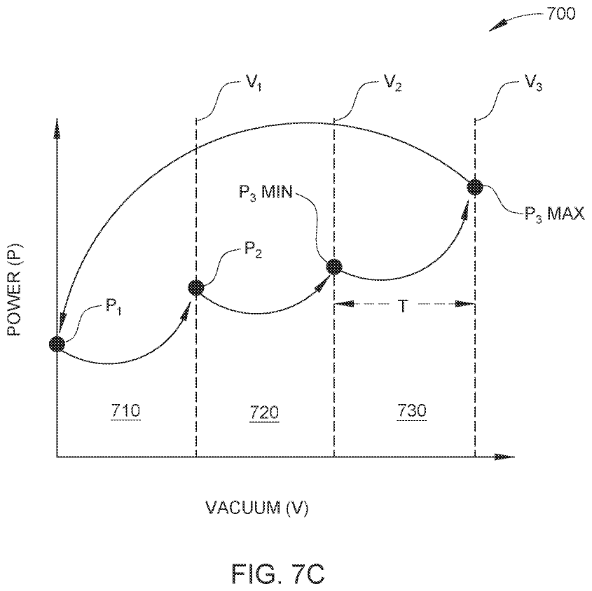

FIG. 7A is a graph 700 which depicts a first example of a “power reset” function. The user may elect to enable a “power reset” option. The “power reset” function prevents power stagnation at high occlusion and high vacuum levels. For example, a power reset may occur when V₁ is 125 mmHg, V2 is 250 mmHG, and the max power setpoint is 500 mmHg. When the power reaches the maximum power setpoint P_{3M}AX for a predetermined period of time, the power will automatically reset to a lower power level. The predetermined period of time may be selected by the user or the predetermined period of time may be inherent in the automated phacoemulsification algorithm. If the predetermined period of time is inherent in the algorithm, the time may depend on the occlusion level.

The “power reset” function aids in breaking up the cataract efficiently as well as allow particle tumbling at the phacoemulsification tip. Alternating between maximum power and resetting to lower powers could also aid in repositioning the particle at the tip of the handpiece 110 and aid in enhanced particle grab and holdability by the handpiece 110, all while remaining cognizant of overall power usage. As shown in FIG. 7A, the power may reset to the third minimum power P_{3MIN} after reaching the maximum power P_{3M}AX for the predetermined period of time. After resetting to the third minimum power P_{3MIN}, the power may incrementally increase back up to the maximum power P_{3MAX}. The power may continuously cycle in this way during automated phacoemulsification.

In FIGS. 7A, 7B, and 7C, sections 710, 720, and 730 define the different vacuum levels detected in the aspiration line based on different levels of occlusion that may be encountered during a phacoemulsification surgery where a user may select at least one vacuum threshold V. In another example, the vacuum thresholds V₁ and V₂ may be determined by the system where those thresholds would be lesser than the user defined maximum vacuum setpoint V₃. Depending on the vacuum or occlusion level, whether in 710, 720, or 730, different power levels P₁, P₂, or P₃ would be applied respectively in either panel mode or linear mode.

FIGS. 7B and 7C illustrate graphs which depict alternative examples of the “power reset” function. These examples have the same purpose and function as the “power reset” shown in FIG. 7A. However, as shown in FIG. 7B, the power may reset to the second power P₂. As shown in FIG. 7C, the power may reset to the first power P₁. The power level may increase (e.g., P₁ to P₂) based on the real-time vacuum level.

A user is able to select from different “power reset” options prior to surgery. For example, the user may select “off,” wherein the “power reset” is disabled and no reset will occur; “low,” wherein the “power reset” will reset to the third minimum power P_{3MIN}; “medium,” wherein the “power reset” will reset to the second power P₂; or “high,” wherein the “power reset” will reset to the first power P₁. The different “power reset” levels provide surgeons with different options as to how much or how little the surgeon would like to reset the power while at high occlusion and vacuum levels when using automated phacoemulsification.

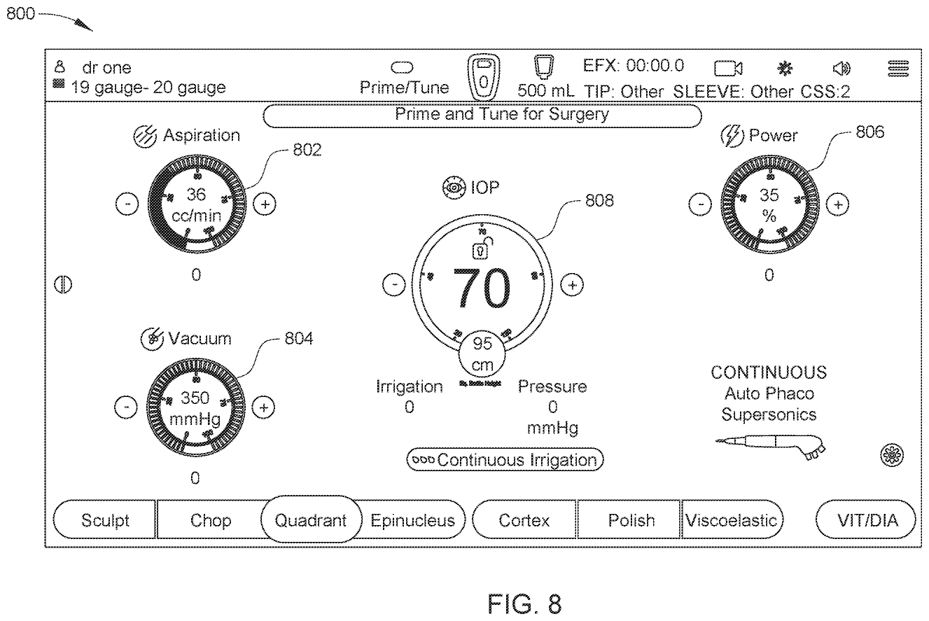

FIG. 8 shows an example graphical user interface (GUI) 800 when automated phacoemulsification is enabled. During automated phacoemulsification, the GUI may display various parameters, including aspiration levels 802, vacuum levels 804, power levels 806, and IOP 808. Automated phacoemulsification parameters may be inputted or selected by the user via the GUI.

The user may be presented with suggested prefilled parameters. Alternatively, the user may be presented with parameters previously saved by the user. In one example, the parameter may include, but is not limited to, max power setpoint, max aspiration setpoint, max vacuum setpoint, vacuum threshold 2, and/or power ramp time. The parameters inputted via the GUI and stored by the surgical console 102 are applied automatically when the automated phacoemulsification algorithm is activated during the procedure or surgery.

The max power setpoint may be the maximum power percentage (from 0-100%) output through the handpiece when operating in the third zone 230. The max aspiration setpoint may be the maximum aspiration flow, from 0-80 cc/minute output through the handpiece. The max vacuum setpoint may be the maximum, from 0-600 mmHG, pull in the aspiration line to pull and remove lens particles from the eye. For example, the vacuum threshold 2 described in FIG. 3 above may be the threshold that dictates the vacuum level that signifies a “full occlusion,” which signals the system to apply more power starting from 35% of the max power setpoint to the max power setpoint. Power ramp time may specify how fast the power will ramp up section 330 (from 35% of the max power setpoint to the max power setpoint). The ramp time may have three options—0.5 seconds, 1 second, and 2 seconds.

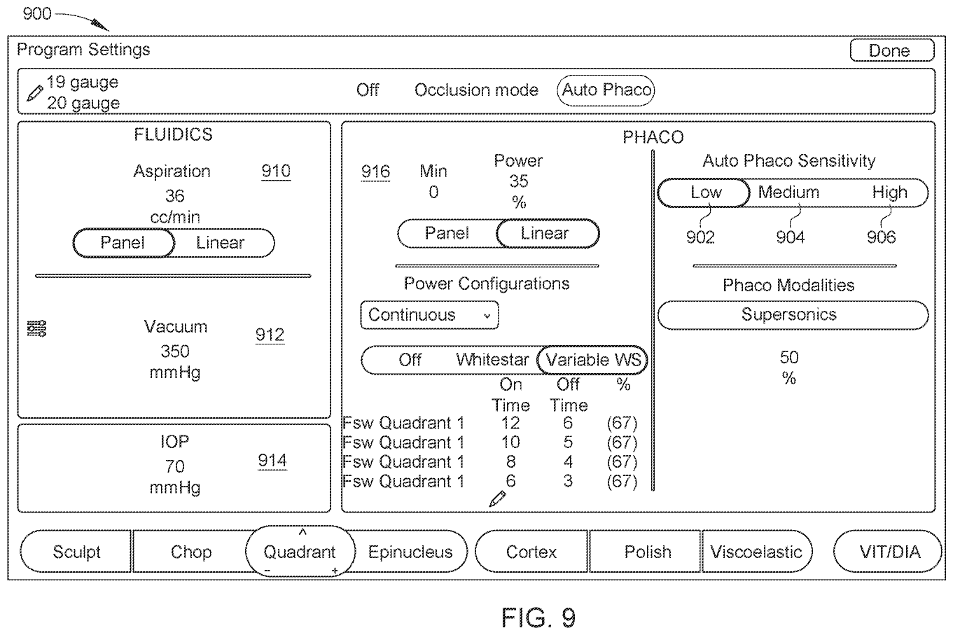

FIG. 9 shows an example GUI 900 for the automated phacoemulsification sensitivity settings. As shown in FIG. 9, the user can select low sensitivity 902, medium sensitivity 904, or high sensitivity 906. Further, the user may adjust settings related to fluidics 910, vacuum 912, IOP 914, and Power 916.

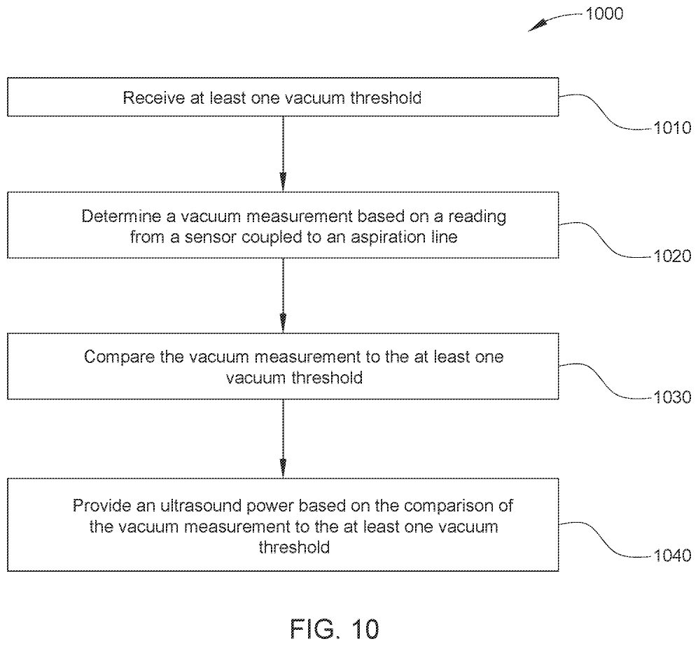

FIG. 10 is a flow chart of a computer-based automated phacoemulsification method 1000. The components and functions involved in the computer-based automated phacoemulsification method include the same details and examples described above. The method 1000 is used to automatically deliver ultrasound power and/or aspiration to a handpiece during a phacoemulsification procedure or surgery based on pre-selected and/or pre-defined parameters and a vacuum measurement based on a sensor coupled with the aspiration line or irrigation line.

In step 1010, at least one vacuum threshold is received. The at least one vacuum threshold may be selected by a user via the GUI. Alternatively, at least one of the vacuum thresholds may be defined by the system algorithm. For example, the user may define a maximum vacuum threshold, while the algorithm defines at least one other vacuum threshold based on the maximum vacuum threshold. The at least one vacuum threshold designates different occlusion levels.

In step 1020, a vacuum measurement is determined based on a reading from the sensor coupled with the aspiration line of the phacoemulsification system. The vacuum measurement is determined during the phacoemulsification procedure in real-time.

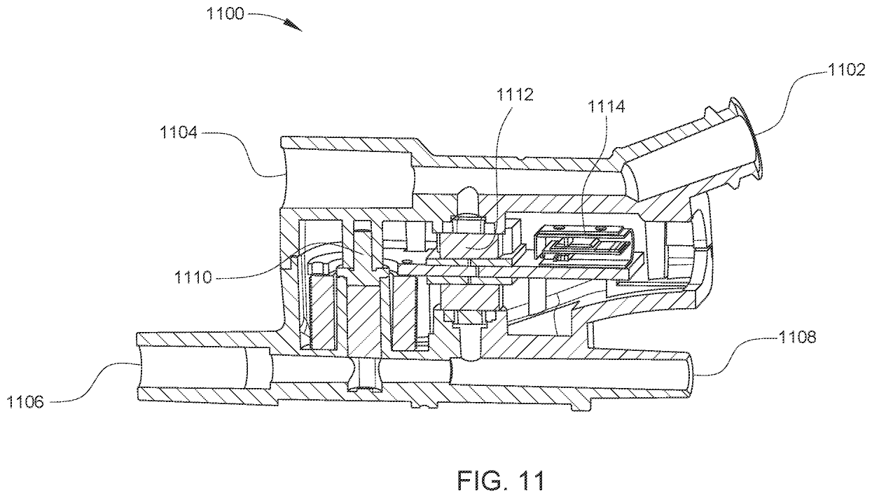

FIG. 11 is a diagram illustrating an exemplary example of an anti-vacuum surge (AVS) module (or chamber stability system (CSS)) 1100. In one example, the system 100 may include the AVS module 1100. The AVS module 1100 may be located between the handpiece 110 and surgical console 102. The AVS module 1100 may be coupled with the distal end of the irrigation line 113 and aspiration line 116 behind the handpiece 110 or in close proximity to the proximal end of the handpiece, (e.g., 4 inches (101.6 mm) from the proximal end of the handpiece).

The AVS module 1100 may include an irrigation-in port 1102, irrigation-out port 1004, aspiration-in port 1106, and aspiration-out port 1108. The irrigation fluid may flow from the irrigation fluid source 112 into the AVS 1100 through the irrigation-in port 1102 and out of the AVS 1100 through the irrigation-out port 1104. The aspiration fluid may flow from the eye through handpiece 110 into the AVS through the aspiration-in port 1106 and out of the AVS 1100 through the aspiration-out port 1108. The AVS 1000 may further include a valve 1110 (e.g., a solenoid valve), sensor 1112 (e.g., pressure or flow), and other electronics 1114. In another example, the AVS may have one or more sensors coupled with the irrigation side.

The AVS module 1100 may be used to detect intraocular pressure (IOP), flow (irrigation & aspiration), and reduce post occlusion surge. The valve 1110 can be activated to close the aspiration line when the sensor 1112 senses a sudden drop in vacuum at a certain rate. When the valve is closed, the power that is delivered below the first threshold (as described in FIGS. 3, 4, and/or 5), may be zero or a minimal power level.

In automated phacoemulsification mode, the aspiration sensor readings are compared to the vacuum threshold level and the power is adjusted accordingly. For example, the aspiration sensor readings may be sent to a fluidics controller located inside the surgical console 102. The system compares the vacuum levels to the designated threshold levels and provides power based on the comparison. The aspiration and irrigation sensor reading may be available to the surgeon via the GUI.

Referring back to FIG. 10, in step 1030, the vacuum measurement is compared to the at least one vacuum threshold. The vacuum measurement is compared to the vacuum threshold by determining whether the measurement is less than or greater than each of the at least one vacuum thresholds. The comparison is used to determine the level of occlusion at the tip of the handpiece of the phacoemulsification system.

In step 1040, an ultrasound power is provided to the handpiece of the phacoemulsification system based on the comparison of the vacuum measurement to the at least one vacuum threshold. The amount or level of ultrasound is determined based on whether the vacuum measurement is greater than or less than the at least one vacuum threshold. If the vacuum measurement is equal to the threshold, the ultrasound power may the power level set prior to reaching the threshold. For example, in FIG. 4, if the vacuum measurement is equal to V₁, the ultrasound power may be P₁.

The amount or level is set by the user or system algorithm prior to surgery, but may also be set or adjusted during surgery. The profile and mode of the ultrasound power may also be selected by the user or system algorithm and automatically applied during the procedure. The system may also apply other parameters, such as aspiration, based on the comparison of the vacuum measurement to the at least one vacuum threshold. The aspiration may be selected by a user or system algorithm prior to the procedure.

Steps 1020, 1030, and 1040 may be repeated throughout the phacoemulsification procedure.

Automated phacoemulsification may also be used with surgical systems that utilize one or more pumps, e.g., a peristaltic pump, progressive cavity pump and/or venturi pump. For example, in a dual pump system with a peristaltic pump and a venturi pump, when the surgeon is in FP3, the venturi pump may be leveraged as a “vacuum boost” when the vacuum level is in section 1 and section 2. This may help increase followability and draw particles to the phacoemulsification tip during surgery. After the particles are purchased and create a high state of occlusion, the vacuum level will reach section 3, where the venturi pump will no longer be activated. The peristaltic pump may then be used for maintaining vacuum until the vacuum level reduces after the particle at the tip is emulsified and aspirated.

### EXAMPLES

### Example 1

A computer-based surgical support method, comprising: receiving at least one vacuum threshold; determining a vacuum measurement based on a reading from a sensor (118) coupled with an aspiration line (116); comparing the vacuum measurement to the at least one vacuum threshold; and providing an ultrasound power based on the comparison of the vacuum measurement to the at least one vacuum threshold.

### Example 2

The method of example 1, wherein the at least one vacuum threshold is a first vacuum threshold, and further comprising: receiving a second vacuum threshold; comparing the vacuum measurement to the second vacuum threshold; and providing a second ultrasound power based on the comparison of the vacuum measurement to the second vacuum threshold wherein providing the second ultrasound power based on the comparison of the vacuum measurement to the second vacuum threshold includes providing the second ultrasound power if the vacuum measurement is greater than the second vacuum threshold.

### Example 3

The method of any of examples 1 to 2, wherein a user inputs the at least one vacuum threshold.

### Example 4

The method of any of examples 1 to 3, wherein the at least one vacuum threshold is based on at least one of a tip size of the needle, an irrigation sleeve size, an intraocular pressure, aspiration flow, and an irrigation flow.

### Example 5

The method of any one of the examples 1 to 4, wherein the vacuum threshold is based on a sensitivity level.

### Example 6

The method of example 5, wherein a user inputs the sensitivity level.

### Example 7

The method of any of the examples 1 to 6, wherein providing the ultrasound power based on the comparison of the vacuum measurement to the at least one vacuum threshold includes providing a first ultrasound power if the vacuum measurement is less than the at least one vacuum threshold and providing a second ultrasound power if the vacuum measurement is greater than the at least one vacuum threshold.

### Example 8

The method of example 7, wherein the second ultrasound power is greater than the first ultrasound power.

### Example 9

The method of any of examples 7 to 8, wherein the second ultrasound power includes an initial ultrasound power and a maximum ultrasound power, and the second ultrasound power linearly increases from the initial ultrasound power to the maximum ultrasound power over a predetermined period of time.

### Example 10

The method of example 9, wherein the second ultrasound power decreases to a lower power when the second ultrasound power is at the maximum ultrasound power for a predetermined period of time.

### Example 11

The method of example 7, wherein the second ultrasound power includes an initial ultrasound power and a maximum ultrasound power, and the second ultrasound power linearly increases from the initial ultrasound power to the maximum ultrasound power as the vacuum measurement increases.

### Example 12

The method of example 7, wherein the second ultrasound power includes an initial ultrasound power and a maximum ultrasound power, and the second ultrasound power linearly increases from the initial ultrasound power to the maximum ultrasound power is based on a predetermined period of time.

### Example 13

The method of any one of the examples 1 to 12, wherein a user inputs the ultrasound power prior to performing an operation.

### Example 14

The method of any one of the examples 1 to 13, further including providing an aspiration boost based on the comparison of the vacuum measurement to the at least one vacuum threshold.

### Example 15

The method of example 14, wherein providing the aspiration boost based on the comparison of the vacuum measurement to the vacuum threshold includes providing a first aspiration boost if the vacuum measurement is less than the at least one vacuum threshold and providing a second aspiration boost if the vacuum measurement is greater than the at least one vacuum threshold.

### Example 16

The method of example 15, wherein the second aspiration boost is less than the first aspiration boost.

### Example 17

The method of example 14, wherein a user inputs the aspiration boost prior to performing an operation.

### Example 18

The method of any one of the examples 1 to 17, wherein the at least one vacuum threshold designates different stages of occlusion.

### Example 19

The method of any of the example 1 to 18, wherein the ultrasound power is a percentage of a max power setpoint.

### Example 20

A surgical system (100) comprising: a handpiece (110); an aspiration line (116) coupled with the handpiece; a sensor (118, 112) communicatively coupled with the aspiration line (116); and a surgical console (102) communicatively coupled to with the handpiece (110), the surgical console (102) configured to provide a predetermined ultrasound power to the handpiece (110) based on a comparison of a measured vacuum level and at least one vacuum threshold, the measured vacuum level is based on a reading from the sensor (118, 112).

### Example 21

The surgical system of any one of examples 19 to 20, wherein the vacuum threshold is based on at least one of a tip size of the needle, an irrigation sleeve size, an intraocular pressure, aspiration flow, and an irrigation flow.

### Example 22

The surgical system of any one of the examples 19 to 21, wherein the vacuum threshold is based on a sensitivity level.

### Example 23

The surgical system of any one of the examples 19 to 22, wherein a user inputs the sensitivity level.

### Example 24

The surgical system of any of the examples 19 to 23, further including a foot pedal (104, 200), wherein the predetermined ultrasound power is provided when the foot pedal is adjusted to a specified position.

### Example 25

The surgical system of any of the examples 19 to 24, wherein a user inputs the at least one vacuum threshold.

### Example 26

The surgical system of any of the examples 19 to 25, wherein a first ultrasound power is provided if the measured vacuum level is less than the at least one vacuum threshold and a second ultrasound power is provided if the measured vacuum level is greater than the at least one vacuum threshold.

### Example 27

The surgical system of example 26, wherein the second ultrasound power is greater than the first ultrasound power.

### Example 28

The surgical system of example 27, wherein the second ultrasound power includes an initial ultrasound power and a maximum ultrasound power, and the second ultrasound power linearly increases from the initial ultrasound power to the maximum ultrasound power as the vacuum measurement increases.

### Example 29

The surgical system of example 28, wherein the second ultrasound power includes an initial ultrasound power and a maximum ultrasound power, and the second ultrasound power linearly increases from the initial ultrasound power to the maximum ultrasound power is based on a predetermined period of time.

### Example 30

The surgical system of example 29, wherein the second ultrasound power decreases to a lower power when the second ultrasound power is at the maximum ultrasound power for a predetermined period of time.

### Example 31

The surgical system of example 30, wherein the second ultrasound power includes an initial ultrasound power and a maximum ultrasound power, and the second ultrasound power linearly increases from the initial ultrasound power to the maximum ultrasound power as the vacuum measurement increases.

### Example 32

The surgical system of any of the exampled 20 to 31, wherein the surgical console includes a graphical user interface (800, 900) configured to receive a user input.

### Example 33

The surgical system of example 32, wherein a user inputs the predetermined ultrasound power prior to performing an operation.

### Example 34

The surgical system of any of the examples 20 to 33, wherein the surgical console is further configured to provide an aspiration boost based on the comparison of the measured vacuum level to the at least one vacuum threshold.

### Example 35

The surgical system of example 34, wherein a first aspiration boost is provided if the measured vacuum level is less than the at least one vacuum threshold and a second aspiration boost is provided if the measured vacuum level is greater than the at least one vacuum threshold.

### Example 36

The surgical system of example 35, wherein the second aspiration boost is less than the first aspiration boost.

### Example 37

The surgical system of example 36, wherein a user inputs the aspiration boost prior to performing an operation.

### Example 38

The surgical system of example 34, wherein a first aspiration boost is provided if the measured vacuum level is less than the at least one vacuum threshold and a second aspiration boost is provided if the measured vacuum level is greater than the at least one vacuum threshold.

### Example 39

The surgical system of any of the exampled 20 to 38, wherein the at least one vacuum threshold designates different stages of occlusion.

### Example 40

The surgical system of any of the exampled 20 to 39, wherein the ultrasound power is a percentage of a max power setpoint.

It should be understood that many variations are possible based on the disclosure herein. Although features and elements are described above in particular combinations, each feature or element can be used alone without other features and elements or in various combinations with or without other features and elements.

What is claimed is:

1. A computer-based surgical support method, comprising:
receiving at least one vacuum threshold;
determining a vacuum measurement based on a reading from a sensor coupled with an aspiration line;
comparing the vacuum measurement to the at least one vacuum threshold; and
providing an ultrasound power based on the comparison of the vacuum measurement to the at least one vacuum threshold.

2. The method of claim 1, wherein the at least one vacuum threshold is a first vacuum threshold, and further comprising:
receiving a second vacuum threshold;
comparing the vacuum measurement to the second vacuum threshold; and
providing a second ultrasound power based on the comparison of the vacuum measurement to the second vacuum threshold;
wherein providing the second ultrasound power based on the comparison of the vacuum measurement to the second vacuum threshold includes providing the second ultrasound power if the vacuum measurement is greater than the second vacuum threshold.

3. The method of claim 1, wherein the vacuum threshold is based on at least one of a tip size of the needle, an irrigation sleeve size, an intraocular pressure, aspiration flow, and an irrigation flow.

4. The method of claim 1, wherein the vacuum threshold is based on a sensitivity level.

5. The method of claim 1, wherein providing the ultrasound power based on the comparison of the vacuum measurement to the at least one vacuum threshold includes providing a first ultrasound power if the vacuum measurement is less than the at least one vacuum threshold and providing a second ultrasound power if the vacuum measurement is greater than the at least one vacuum threshold.

6. The method of claim 5, wherein the second ultrasound power is greater than the first ultrasound power.

7. The method of claim 5, wherein the second ultrasound power includes an initial ultrasound power and a maximum ultrasound power, and the second ultrasound power linearly increases from the initial ultrasound power to the maximum ultrasound power over a predetermined period of time.

8. The method of claim 7, wherein the second ultrasound power decreases to a lower power when the second ultrasound power is at the maximum ultrasound power for a predetermined period of time.

9. The method of claim 5, wherein the second ultrasound power includes an initial ultrasound power and a maximum ultrasound power, and the second ultrasound power linearly increases from the initial ultrasound power to the maximum ultrasound power as the vacuum measurement increases.

10. The method of claim 5, wherein the second ultrasound power includes an initial ultrasound power and a maximum ultrasound power, and the second ultrasound power linearly increases from the initial ultrasound power to the maximum ultrasound power is based on a predetermined period of time.

11. The method of claim 1 further including providing an aspiration boost based on the comparison of the vacuum measurement to the at least one vacuum threshold.

12. The method of claim 11, wherein providing the aspiration boost based on the comparison of the vacuum measurement to the vacuum threshold includes providing a first aspiration boost if the vacuum measurement is less than the at least one vacuum threshold and providing a second aspiration boost if the vacuum measurement is greater than the at least one vacuum threshold.

13. The method of claim 12, wherein the second aspiration boost is less than the first aspiration boost.

14. The method of claim 1, wherein the ultrasound power is a percentage of a max power setpoint.

15. A surgical system comprising:
a handpiece;
an aspiration line coupled with the handpiece;
a sensor communicatively coupled with the aspiration line; and
a surgical console communicatively coupled with the handpiece, the surgical console configured to provide a predetermined ultrasound power to the handpiece based on a comparison of a measured vacuum level and at least one vacuum threshold, the measured vacuum level is based on a reading from the sensor.

16. The surgical system of claim 15, wherein the vacuum threshold is based on at least one of a tip size of the needle, irrigation sleeve size, an intraocular pressure, aspiration flow, and an irrigation flow.

17. The surgical system of claim 15, wherein the vacuum threshold is based on a sensitivity level.

18. The surgical system of claim 15, further including a foot pedal, wherein the predetermined ultrasound power is provided when the foot pedal is adjusted to a specified position.

19. The surgical system of claim 15, wherein a user inputs the at least one vacuum threshold.

20. The surgical system of claim 15, wherein a first ultrasound power is provided if the measured vacuum level is less than the at least one vacuum threshold and a second ultrasound power is provided if the measured vacuum level is greater than the at least one vacuum threshold.

21. The surgical system of claim 20, wherein the second ultrasound power is greater than the first ultrasound power.

22. The surgical system of claim 21, wherein the second ultrasound power includes an initial ultrasound power and a maximum ultrasound power, the second ultrasound power configured to linearly increase from the initial ultrasound power to the maximum ultrasound power over a predetermined period of time.

23. The surgical system of claim 22, wherein the second ultrasound power decreases to a lower power when the second ultrasound power is at the maximum ultrasound power for a predetermined period of time.

24. The surgical system of claim 23, wherein the second ultrasound power includes an initial ultrasound power and a maximum ultrasound power, and the second ultrasound power linearly increases from the initial ultrasound power to the maximum ultrasound power as the vacuum measurement increases.

25. The surgical system of claim 23, wherein the second ultrasound power includes an initial ultrasound power and a maximum ultrasound power, and the second ultrasound power linearly increases from the initial ultrasound power to the maximum ultrasound power is based on a predetermined period of time.

26. The surgical system of claim 16, wherein the surgical console is further configured to provide an aspiration boost based on the comparison of the measured vacuum level to the at least one vacuum threshold.

27. The surgical system of claim 26, wherein a first aspiration boost is provided if the measured vacuum level is less than the at least one vacuum threshold and a second aspiration boost is provided if the measured vacuum level is greater than the at least one vacuum threshold.

28. The surgical system of claim 27, wherein the second aspiration boost is less than the first aspiration boost.

29. The surgical system of claim 28, wherein the ultrasound power is a percentage of a max power setpoint.
